(19) United States
(12) Patent Application Publication
(10) Pub. No.: US 2003/0153863 A1
(43) Pub. Date: Aug. 14, 2003

(54) Implant system for glaucoma surgery

(76) Inventor: Anilbhai S. Patel, Arlington, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/074,975

(22) Filed: Feb. 13, 2002

(51) Int. Cl. A61M 5/00; A61F 2/04

(52) U.S. Cl. 604/8; 623/23.64; 623/23.71

(57) ABSTRACT

An implant system which facilitates and improves the non-penetrating deep sclerectomy/canalostomy surgery by assured maintenance of the scleral aqueous filled space by a basic permanent chamber which can be easily connected into the opening of the Schlemm's canal by adjustable connecting tubes. This implant system also provides surgical options for penetrating through Descemet's membrane as well as for outflow into the subconjunctival space though the superficial scleral layer.

### BACKGROUND OF THE INVENTION

This invention relates generally to the field of glaucoma surgery and, more particularly, to an implant for glaucoma surgery.

Glaucoma is a disease of the eye that causes blindness in millions of people. The disease is not understood totally, and many do not even categorize glaucoma as a single disease. Glaucoma is more accurately identified as an optic neuropathy characterized by a specific pattern of optic nerve head and visual field damage, which are the end result of a number of different conditions that can affect the eye. Elevated intraocular pressure (IOP) is an important risk factor for the development or progression of glaucomatous damage. Elevated IOP can be caused either by elevated production of aqueous humor in the eye or by restricted flow of the aqueous from the eye. There are several pharmaceutical treatments for glaucoma, including beta-blockers and prostaglandin analogs but in extreme cases, surgical intervention to increase aqueous outflow is used.

There are several and confusing terms used for the glaucoma surgery involving deep sclerectomy. They include viscocanalostomy, non-penetrating deep sclerectomy, and penetrating conversions of these operations. Deep sclerectomy is the term commonly used for the non-penetrating glaucoma surgery in which deep layers of sclera are removed after the deep scleral delamination down to the Descemet's membrane without penetrating into the anterior chamber. This delamination is carried out carefully up to and including the transition zone into the anterior trabecula up to about 1 mm in front of Schwalbe's line, resulting in a window for the controlled outflow of the aqueous humor through the trabecula-Descemet's membrane and the controlled reduction in the IOP, thus avoiding post-surgical hypotony. Schlemm's canal is also excised for the width of the removed deep sclera. Also removed if necessary is the juxta-canalicular trabecular meshwork tissue with a fine forceps, leaving behind the innermost bulk of the trabecular meshwork. This is done to assure sufficient percolation of the aqueous humor. This additional step has also been called “ab externo trabeculectomy” and is also considered non-penetrating because no direct entry in the anterior chamber is done as is the case with trabeculectomy which removes the entire trabecular meshwork.

Earlier, it was thought that the deep sclerectomy created scleral space may act postoperatively as an aqueous reservoir and may either prevent a subconjunctival filtration bleb and thus bleb-related late complications including endophthalmitis. In reality, the surgically created space was not maintained and the outflow through the relatively loose flap into subconjunctival space resulted in subconjunctival bleb formation. Also, the fibrosis of the scleral space lost the outflow passage and thus failed the operation for many cases.

Attempts were made to maintain space by a hydrogel device in first implanted in Russia which failed because of a pronounced tendency for capsulation and thus stoppage of the flow. A collagen device has also been used, achieving better results. But even with the collagen device, the subconjunctival outflow results in the bleb formation in successful surgeries. Some have reported that the collagen device increases the success rate by reduction of scleral flap fibrosis and bleb fibrosis and also reduces the overall complications of non-penetrating deep sclerectomy. Some also believe that the collagen device reduces the incidents of post-operative immediate hypotony. The long term stability of the collagen or other biopolymer device is of concern due to the eventual dissolving of the device by the environmental enzymatic, hydrolytic and other biological processes. Thus, interest for devices made from non-resorbable materials is on-going.

Viscocanalostomy was an attempt to improve the above described non-penetrating deep sclerectomy by additional steps of placing a viscoelastic substance into the space as well as in to the openings of the excised Schlemm's canal. Some report that this step not only keeps the canal open (which otherwise may collapse) but also may create additional microperforation across the juxta-canalicular trabeculum within the canal. The superficial remaining scleral flap is tightly sutured. This surgery thus attempts to keep the scleral space by preventing fibrotic closure of it by use of viscoelastics as well as attempts to establish natural outflow pathway through the Schlemm's canal, which is not only dilated along several millimeters of its length, but is also prevented from fibrotic closing by the viscoelastics. In a successful viscocanalostomy procedure, no outflow into the subconjunctival space occurs and thus no bleb formation occurs. If the flap is leaking, then a bleb may occur to varying degree. The viscoelastics substances used thus far are biopolymers such as sodium hyaluronate (HA) or a mixture of HA with chondroitin sulfate (CS). All of these biopolymers eventually either flow out or are re-absorbed. In addition, some surgeons have found it difficult to locate Schlemm's canal and thus, they removed this step and allowed the outflow through loosely sutured superficial scleral flap into the subconjunctival space forming a bleb, essentially converting the operation into a deep sclerectomy.

While performing either deep sclerectomy or viscocanalostomy, unintended accidental surgical penetration through Descemet's membrane into the anterior chamber often occurs resulting in the procedure becoming a penetrating operation. Some surgeons also find it difficult to carry out the scleral delamination down to the exposure of Descemet's membrane. To ensure adequate flow through Descemet's, manual, uncontrolled punctures are made. In addition, if adequate post-operative flow is not achieved, a secondary procedure involving puncturing the trabecular meshwork from within the anterior chamber is performed using a gonioscopy contact lens and a Nd-YAG laser.

Known complications of traditional penetrating trabeculectomy include: i) hypotony (excessive IOP drop) with resulting flat anterior chamber; ii) an increased rate of cataract progression; iii) conjunctival fibrosis and resulting decrease in aqueous outflow; iv) late thinning of the filtering bleb; v) endophthalmitis; vi) iris incarceration into the filtering site; vii) vitreous herniation into filtration site; viii) uveitis; and ix) vitreous loss.

Known advantages of non-penetrating deep sclerectomy with subconjunctival bleb formation include the reduction or elimination of items i-iii and v-vii listed above. Disadvantages include: a) late thinning of the filtering bleb; and b) fibrosis of the scleral space and conjunctival outflow path.

Known advantages of viscocanalostomy include elimination of all bleb-related and conjunctival fibrosis-related complications of deep sclerectomy, but the surgery is difficult and has been reported to fail in many cases because of fibrosis of the scleral space or the canal opening and the canal outflow path.

Therefore, a need continues to exist for an implantable, non-absorbable device for use during glaucoma surgery.

### BRIEF SUMMARY OF THE INVENTION

The present invention improves upon the prior art by providing an implant system which facilitates and improves the non-penetrating deep sclerectomy/canalostomy surgery by assured maintenance of the scleral aqueous filled space by a basic permanent chamber which can be easily connected into the opening of the Schlemm's canal by disclosed adjustable connecting tube. This adjustability is required to address the need of variable and unpredictable anatomical location of the Schlemm's canal from patient to patient. In the event canal can not be found no such tube will be placed and the superficial flap will be not tightly closed and the plug from the top of the implant chamber will be removed thus allowing passage through the opened hole previously kept closed by the plug. Thus subconjunctival outflow with bleb formation similar to the deep sclerectomy surgery will be a fall back option, still maintaining the aqueous filled scleral lake in the chamber. If such outflow is judged to be inadequate as demonstrated by inadequate bleb formation, then the superficial flap can be opened up and a piercing tube can replace a provision of the plugged opening into the roof of the implanted chamber after removing the plug. This will result in adequate subconjunctival outflow and bleb formation.

In the event if the surgeon finds it difficult to expose the Descemet's membrane or accidental penetration has occurred, I have an additional implant component in the form of a base plate with a penetrating canula into the anterior chamber with appropriate lumen to convert into a controlled penetrating surgery, still retaining preferably the outflow into the Schlemm's canal by the above described chamber reservoir which will fit into the grooves of the bottom plate implant. Thus there will be still no bleb and the outflow will be through the natural route of the canal. Again here to if the canal cannot be found the option of loose flap suturing for the subconjunctival route with bleb formation is still available for the completion of the surgery. Also the plug from the top of the implant chamber will be removed thus allowing passage though the opened hole previously kept closed by the plug. If such outflow is judged to be inadequate as demonstrated by inadequate bleb formation, then the superficial flap can be opened up and a piercing tube can replace a provision of the plugged opening into the roof of the implanted chamber after removing the plug. This will result in adequate subconjunctival outflow and bleb formation.

Finally in the event after successful completion of the surgery, if postoperatively it is found that the natural outflow through the canal as successfully established is not adequate or lost then, only the superficial flap needs to be opened up and a piercing tube can replace a provision of the plugged opening into the roof of the implanted chamber after removing the plug. This will result in subconjunctival outflow and bleb formation. This contingency provision for additional outflow will avoid surgical removal of tubes already established into the canal. This provision also can test whether the limitation is of the outflow path through the canal or the filtering across the trabeculo-Descemet's membrane. If such improved outflow does not restore he desired reduction of the intraocular pressure, then the filtering needs to be increased either by goniopuncture by Nd-YAG or perhaps placing the base plate implant with penetrating canula provision as described above.

Various components of my implant system will be fabricated using non-resorbable, bio-stable, bio-compatible hard or appropriately soft materials. They may be further coated with appropriate materials to discourage fibrotic or cellular proliferation processes. Initially during the surgery they may also be filled with viscoelastics with or without appropriate amount of antimetabolite such as Mytomycin or 5-FU. The top of the implant and remaining space also may also be coated with antimetabolite Mytomycin or 5-FU and /or at least filled with viscoelastics. All the materials used for the implant system will be non-toxic, non-immunogenic and remain stable in the environment of its use.

In short my implant system offers flexibility of achieving glaucoma surgery with the ideal goal of non-penetrating with natural outflow through Schlemm's canal procedure without any bleb and also offers optimum procedure to various other surgical conversions as required to handle the surgical accidents and/or failure of the optimal procedure and still manage the goal of IOP reduction rather than accept total immediate failure of the surgery. Also unique nonabsorbent aqueous reservoir chamber of my invention provides a pseudo-anterior chamber and no cellular growth into it is expected as is the case with the anterior chamber. In the viscocanalostomy surgical procedure, the use of viscoelastics, because it disappears after a limited duration, leads to possibility of fibrosis and the chamber of my invention totally prevents it. Flexible but permanent outflow tube provision of the invention also maintains the outflow into the Schlemm's canal open unlike temporary opening by viscoelastics which will disappear after a limited duration. Use of collagen or hydrogel implant without the provision of outflow tubes into the canal leads to need of subconjunctival outflow with bleb formation which is not ideal and my system, while will allow such outflow path if necessary, does not require it and provides for a natural outflow into the Schlemm's canal.

Accordingly, one objective of the present invention is to provide an implant sized and shaped to fit within the stromal cavity formed during non-penetrating deep sclerectomy/canalostomy surgery.

Another objective of the present invention is to provide a non-penetrating deep sclerectomy/canalostomy implant that contains a plurality of sidewalls that form a hollow interior.

Still another objective of the present invention is to provide a non-penetrating deep sclerectomy/canalostomy implant that contains a pair of tubular elements sized and shaped to fit within Schlemm's canal.

Still another objective of the present invention is to provide a non-penetrating deep sclerectomy/canalostomy implant that helps prevent fibrotic activity in the void created during non-penetrating deep sclerectomy/canalostomy surgery.

These and other advantages and objectives of the present invention will become apparent from the detailed description and claims that follow.

### BRIEF DESCRIPTION OF THE DRAWING

FIG. 1 is a bottom exploded perspective view of the implant of the present invention

FIG. 2 is a top exploded view of the implant of the present invention

FIG. 3 is a perspective view showing the initial incision made during non-penetrating deep sclerectomy/canalostomy surgery.

FIG. 4 is a perspective view showing the dissection of the deepest scleral fibers thereby exposing Descemet's membrane and Schlemm's canal made during the surgery.

FIG. 5 is a perspective view showing the final surgical site ready to receive the implant of the present invention.

### DETAILED DESCRIPTION OF THE INVENTION

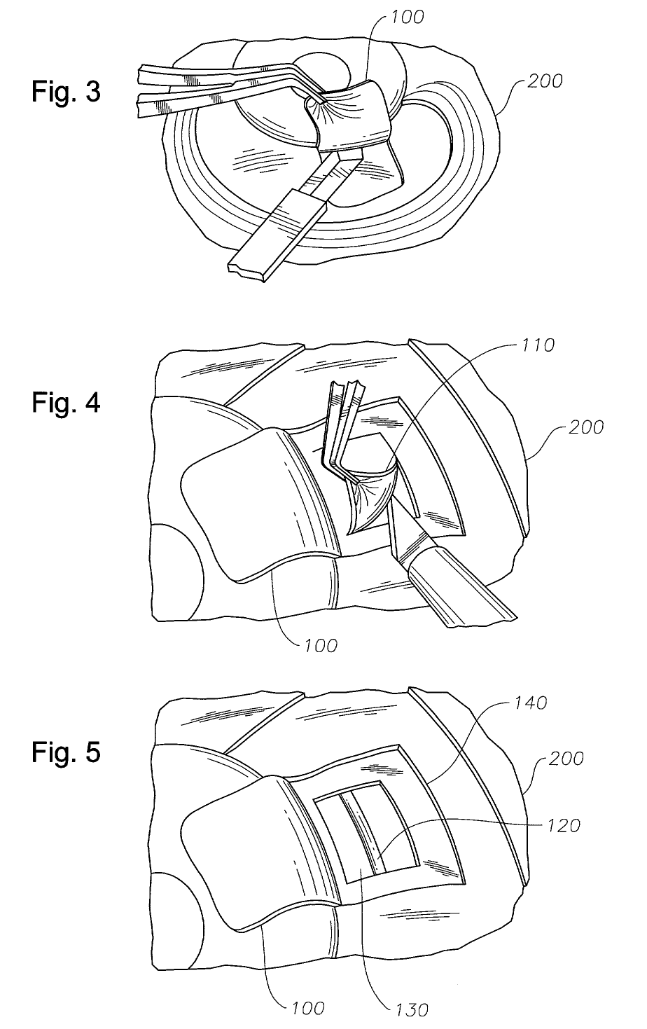

As best seen in FIGS. 3-5, the non-penetrating deep sclerectomy/canalostomy surgical procedure involved with implant 10 of the present invention involves creating a scleral cavity or pocket into which implant 10 is placed. Initially, superficial scleral flap 100 is formed in eye 200. Flap 100 is generally less than one-third of the scleral thick and can extend up to two millimeters into the clear cornea. Flap 100 may be generally 5 millimeters by 6 millimeters but can be made larger or smaller depending upon factors such as the size of the eye or the desired size of implant 10. The sclera is further dissected to remove deep portion of sclera 110 to expose Descemet's membrane 130 leaving a thin layer of deep sclera over the choroid posteriorly. Anteriorly, the dissection is done reaching Schlemm's canal 120 (which is unroofed) and continuing further into corneal stromal tissue to the level of Descemet's membrane 130. The removal of the inner endothelium of Schlemm's canal 120 and juxta-canalicular trabecula can also be performed at this stage using a fine forceps (not shown). The aqueous percolation into the achieved scleral cavity will thus be established. Deep scleral portion 110 can be of any size suitable to allow for implantation of implant 10, but generally will be approximately 4 millimeters by 5 millimeters, but can be made larger or smaller depending upon factors such as the size of the eye or the size of flap 100.

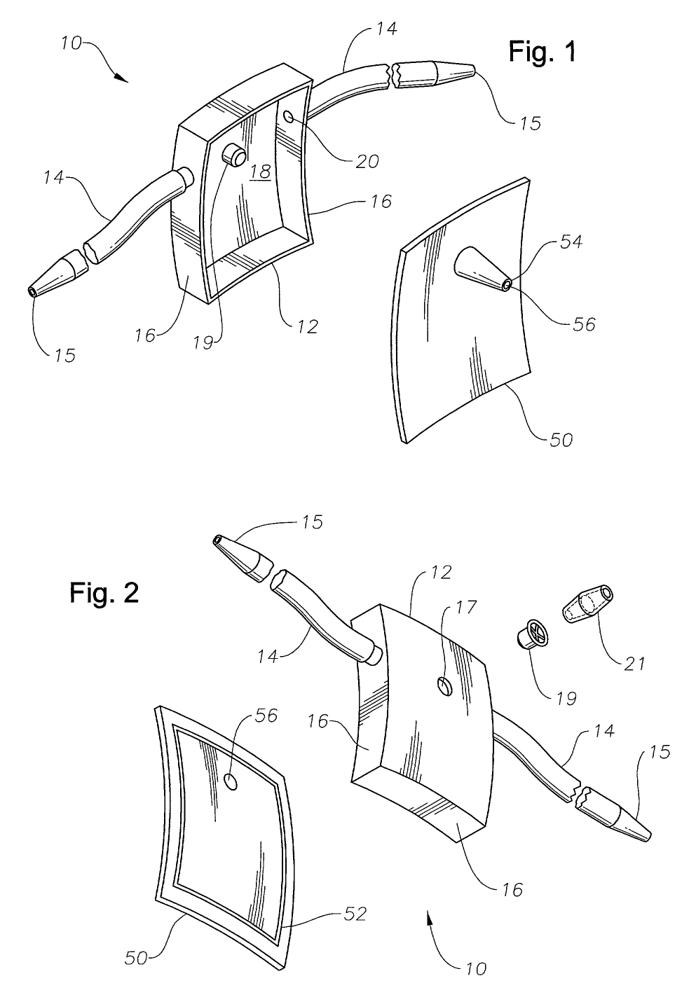

As best seen in FIGS. 1-2, implant 10 of the present invention is designed to be implanted within a void formed in the sclera during non-penetrating deep sclerectomy/canalostomy surgery by the removal of deep sclera portion 110 and includes body 12 and tubular elements 14. Implant 10 preferably is made in multiple components from any suitable biocompatible material, such as polymethylmethacrylate (PMMA), polycarbonate, polyurethane, polyamide, polypropylene, silicone, soft acrylic, hydrogel, stainless steel or titanium. Implant 10 may be coated with any suitable coating to enhance biocompatibility or to help prevent implant 10 from fouling or become clogging with fibrotic growth, such as heparin, mytomycin, 5-FU or other suitable coatings well-known in the art. Body 12 is generally flat or slightly curved to approximate the curvature of the sclera and contains sidewalls 16 that form hollow interior 18. Body 12 may be of any suitable size and shape, such as rectangular, semi-circular or elliptical and between 4 millimeter and 5 millimeters across, but can be made larger or smaller depending upon factors such as the size of the eye or the size of deep scleral portion 110. Body 12 may contain port 17 that communicates with hollow interior 18. Sidewalls 16 preferably are between 100 microns and 200 microns tall. Tubes 14 may have tapered ends 15 and preferably are sized and shaped to fit snugly within the openings in the unexcised portions of Schlemm's canal and contains bores 20 that communicate with hollow interior 18. Tubes 14 may be made adjustable to address the need of variable and unpredictable anatomical location of Schlemm's canal 120 from patient to patient.

Implant 10 may also contain bottom plate 50 having a similar construction as body 12 and containing a circumferential groove 52 sized and shaped so as to allow plate 50 to securely fit onto and be held by sidewalls 16 on body 12. Plate 50 has outwardly tapering fitting 54 having a port 56 that communicates with hollow interior 18 of body 12 when plate 50 is attached to body 12.

In use during non-penetrating deep sclerectomy/canalostomy surgery, void 140 is created in the sclera that exposes Descemet's membrane 130 and Schlemm's canal 120 in the manner described above. Body 12 is place in void 140 so that sidewalls 16 lay perpendicularly to Descemet's membrane 130 and hollow interior 18 is exposed to Descemet's membrane 130. If desired, body 12 may be filled with a viscoelastic substance, such substances being well-known in the art, to help minimize fibrotic adhesions. In addition, the viscoelastic agent may contain a antimetabolite, such as mytomycin or 5-FU. Tapered ends 15 of tubular elements 14 are inserted into the openings in the unexcised portions of Schlemm's canal 120. If desired, the openings in the unexcised portions of Schlemm's canal 120 may be enlarged slightly by the introduction of a viscoelastic substance, such substances being well-known in the art in order to facilitate the introduction of tapered ends 15 into the openings in the unexcised portions of Schlemm's canal 120. Flap 100 is placed over implant 100 and sutured in place. Port 17 in body 12 normally will be sealed by plug 19 so that any fluid entering hollow interior 18 will be contained within body 12. In this manner, aqueous fluid may percolate through Descemet's membrane 130 and enter the openings in the unexcised portions of Schlemm's canal 120 through hollow interior 18, ports 20 in tubular arms 15. In the event that Schlemm's canal 120 can not be found and/or tubes 14 can not be placed in the openings in the unexcised portions of Schlemm's canal 120, flap 100 will be not tightly closed and plug 19 may be removed from body 12 thus allowing passage through port 17, causing subconjunctival outflow with bleb formation similar to the deep sclerectomy surgery. If inadequate bleb formation occurs, then flap 100 may be opened and plug 19 may be removed from port 17 and replaced with piercing plug 21 and flap 100 replaced. Alternatively, even with successful placement of tubes 14 into the openings in the unexcised portions of Schlemm's canal 120, in the event that outflow from interior 18 through ports 20 and into the openings in the unexcised portions of Schlemm's canal 120 becomes blocked or is insufficient to reduce intraocular pressure a sufficient amount, flap 100 may be opened and plug 19 may be removed from port 17 and replaced with piercing plug 21 and flap 100 replaced. Plug 21 allows excess aqueous fluid to exit interior 18 through flap 100 and into the subconjunctival space between the scleral and conjunctiva, thereby forming a subconjunctival bleb.

In the event that percolation through Descemet's membrane 130 is insufficient to relieve the excess intraocular pressure, plate 50 may be used by attaching plate 50 to body 12 and placing the combination of body 12 and plate 50 in void 140 so that fitting 54 punctures Descemet's membrane 130 and projects downwardly into the anterior chamber of eye 200. Port 56 allows for more positive drainage of aqueous fluid from the anterior chamber into hollow interior 18 and out through ports 20 into the openings in the unexcised portions of Schlemm's canal 120. In the event that outflow from interior 18 through ports 20 and into the openings in the unexcised portions of Schlemm's canal 120 becomes blocked or is insufficient to reduce intraocular pressure a sufficient amount, plug 19 may be removed from port 17 and replaced with plug 21. Plug 21 allows excess aqueous fluid to exit interior 18 through flap 100 and into the subconjunctival space between the scleral and conjunctiva, thereby forming a subconjunctival bleb.

This description is given for purposes of illustration and explanation. It will be apparent to those skilled in the relevant art that changes and modifications may be made to the invention described above without departing from its scope or spirit.

I claim:

1. A glaucoma implant, comprising:
a) a body, the body having sidewalls defining a hollow interior; and
b) at least one flexible tube attached to one of the sidewalls, the tube having a port through the sidewall allowing the tube to communicate with the hollow interior.

2. The implant of claim 1 further comprising a bottom plate sized and shaped to attach to the sidewalls.

3. The implant of claim 1 wherein the implant comprises polymethylmethacrylate.

4. The implant of claim 1 wherein the implant comprises polycarbonate.

5. The implant of claim 1 wherein the implant comprises silicone.

6. The implant of claim 1 wherein the implant comprises a soft acrylic.

7. The implant of claim 1 wherein the implant comprises stainless steel.

8. The implant of claim 1 wherein the implant comprises titanium.

9. The implant of claim 1 wherein the implant comprises a hydrogel.

10. The implant of claim 1 wherein the implant comprises polyurethane.

11. The implant of claim 1 wherein the implant comprises polyamide.

12. The implant of claim 1 wherein the implant comprises polypropylene.

13. The implant of claim 2 wherein the implant comprises polymethylmethacrylate.

14. The implant of claim 2 wherein the implant comprises polycarbonate.

15. The implant of claim 2 wherein the implant comprises silicone.

16. The implant of claim 2 wherein the implant comprises a soft acrylic.

17. The implant of claim 2 wherein the implant comprises stainless steel.

18. The implant of claim 2 wherein the implant comprises titanium.

19. The implant of claim 2 wherein the implant comprises a hydrogel.

20. The implant of claim 2 wherein the implant comprises polyurethane.

21. The implant of claim 2 wherein the implant comprises polyamide.

22. The implant of claim 2 wherein the implant comprises polypropylene.

23. The implant of claim 1 wherein the implant further comprises a coating.

24. The implant of claim 2 wherein the implant further comprises a coating.
